(19) United States
(12) Patent Application Publication
(10) Pub. No.: US 2009/0088360 A1
(43) Pub. Date: Apr. 2, 2009

(54) Bath Treatment Compositions and Methods

(75) Inventors: Scott W. Wenzel, Neenah, WI (US); Jeffery Seidling, Neenah, WI (US); Stacy A. Mundschau, Weyauwega, WI (US); Jason Laumer, Appleton, WI (US)

Correspondence Address: DORITY & MANNING, P.A., POST OFFICE BOX 1449, GREENVILLE, SC, 29602-1449, US

(73) Assignee: Kimberly-Clark Worldwide, Neenah, WI (US)

(21) Appl. No.: 11/863,433

(22) Filed: Sep. 28, 2007

(51) Int. Cl. A61K 8/81; A61Q 19/10

(52) U.S. Cl. 510/159

(57) ABSTRACT

The present disclosure is generally directed to a bath treatment kit. The bath treatment kit includes a first formulation and second formulation. The first formulation includes a rheology modifier, the rheology modifier being present in a liquid and capable of thickening an aqueous bath. The second formulation includes a deswelling agent, the deswelling agent capable of thinning an aqueous bath previously thickened by the rheology modifier.

### BACKGROUND

Spa services such as mud baths are increasing in popularity. Mud baths have the benefit of relieving stress and detoxifying the body. However, to date, there have been no offerings that can mimic the experience of a thickened mud soak within the home. There are mud bath products for the home that generally consist of additives which color the water and do not create the consistency of a true mud bath. Additionally, there are bath jellies that also do not create the consistency or characteristics of a true mud bath. See, e.g. U.S. Pat. No. 6,281,177 to Moriyama.

For example, the Moriyama patent describes a gelatinous bath jelly in which the components are in powder form. Powder tends to clump and hydrate from the outside forming balls of unhydrated polymer commonly referred to as “fish eyes.” In addition, sodium polyacrylate powders can be highly cross-linked and form super absorbent material that upon hydrating will also form fish eyes, but tend to hydrate more thoroughly and generally do not contain unhydrated polymer internally. The resultant appearance still is gelatinous and appears as multiple small beads which are cosmetically unappealing and have an unpleasant texture which is slimy, sticky, and does not impart a smooth, creamy spa-like mud bath experience. Powders also tend to be very hydroscopic and absorb water from the environment resulting in powder clumping in packaging. In addition, agitation in a bath must be extremely vigorous for powders to disperse evenly and create a smooth appearance similar to a mud bath, hence the gelatinous nature of the bath jelly of Moriyama.

As the technology stands today, to create a thickened mud bath in the home would require the import of many gallons of mud. Such a mud bath is impractical because it would lead to excessive mess and be difficult to clean up and dispose of.

A need exists for a bath treatment that can be added to a reservoir of water and quickly create a thickened solution that has a mud-like consistency and feel. Furthermore, a need exists for a bath solution that offers the benefits of a traditional mud bath for relieving stress and detoxifying the body while also providing the benefits found in many current bath composition offerings. In this manner, a user could soak his or her feet, hands, and/or body in a thickened mud-like solution while getting the benefit derived from both a mud bath as well as a traditional bath formulation. Finally, a need exists for a bath treatment that can readily drain through conventional drainage systems and can be easily cleaned up.

### SUMMARY

Objects and advantages of the disclosure will be set forth in part in the following description, or may be obvious from the description, or may be learned through the practice of the disclosure.

The present disclosure is generally directed to a bath treatment kit. The bath treatment kit includes a first formulation and second formulation. The first formulation includes a rheology modifier, the rheology modifier being present in a liquid and capable of thickening an aqueous bath. The second formulation includes a deswelling agent, the deswelling agent capable of thinning an aqueous bath previously thickened by the rheology modifier.

In another exemplary embodiment, the present disclosure is generally directed to method of using a bath treatment kit. The method includes adding a first formulation to a water container; the first formulation includes a rheology modifier present in a liquid for thickening a portion of the water in the water container. The method also includes adding a second formulation to a water container, the second formulation including a deswelling agent capable of thinning the water in the water container previously thickened by the rheology modifier.

Other features and aspects of the present disclosure are discussed in greater detail below.

### BRIEF DESCRIPTION OF THE DRAWINGS

A full and enabling disclosure of the present disclosure, including the best mode thereof to one of ordinary skill in the art, is set forth more particularly in the specification, including reference to the accompanying Figures in which:

FIGS. 1 and 2 are graphs that indicate that formulations in accordance with the present disclosure have viscosity that can affect drainage; and

FIG. 3 is a graph of rheology data of water, a bath consisting of 5% RapiThix (sodium polyacrylate and hydrogenated polydecene and trideceth-6) (remaining water), and a bath consisting of 5% RapiThix (remaining water) in which 10% sodium chloride was added.

### DETAILED DESCRIPTION

It is to be understood by one of ordinary skill in the art that the present discussion is a description of exemplary embodiments only, and is not intended as limiting the broader aspects of the present disclosure, which broader aspects are embodied in the exemplary construction.

The present disclosure is generally directed to compositions and methods for bath treatments. In that regard, bath treatments described herein can be added to a reservoir of water and create a thickened solution that has a mud-like consistency and feel. In addition, bath treatments described herein offer the benefits of a traditional mud bath for relieving stress and detoxifying the body while also providing additional benefits found in many current bath offerings.

Initially, as referred to herein the term “bath” refers to water for immersing all or part of the body. The term “mud” refers to a liquid or semi-liquid mixture of water and soil, silt, clay (natural or synthetic) or some combination thereof.

In one embodiment of the present disclosure, a bath treatment kit is disclosed. The bath treatment kit includes a first formulation which includes a rheology modifier and a second formulation that includes a deswelling agent.

Rheology modifiers that swell quickly and efficiently within an aqueous environment and whose thickening properties are triggered, or sensitive, to external factors are preferred in connection with the present disclosure. The present disclosure contemplates predispersion of one or more rheology modifiers in an anhydrous vehicle or utilizing vendor created raw materials that are emulsions that can be dispersed easily into water to form thickened solutions. Such rheology modifiers present in liquid will allow for an even and quick dispersion and create a more mud-like composition.

Suitable rheology modifiers include, but are not limited to, sodium polyacrylate, acrylate copolymers and water and/or oil based dispersions, polymethylmethacrylate, polyacrylamide dispersions and clays such as Laponite, which is a synthetic smectic clay of the hectorite type structure, (manufactured by LaPorte, and distributed by RTD Chemicals). In certain embodiments, a preferred rheology modifier is Aristoflex AVC (Ammonium Acryloyldimethyltaurate/VP Copolymer manufactured by Clariant).

The formulations of the present disclosure can contain one or more rheology modifiers, present from about 1% to about 99% by weight of the thickening/swelling formulation, more specifically, from about 4% to about 97% by weight of the formulation. However, the amounts of rheology modifier can vary depending upon the polymer utilized and whether it is a suspension or vendor supplied raw material.

In certain embodiments, the rheology modifier formulation is about 0.25% to about 10% by weight of the overall liquid capacity of the vessel that contains the water to be thickened, more specifically, 0.75% to about 7% by weight, more specifically from about 1.25% to about 3% by weight. Nonlimiting classes of rheology modifiers include those selected from the following:

a) Carboxylic Acid Polymers

These polymers are crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol. Polymers useful in the present disclosure are more fully described in U.S. Pat. No. 5,087,445, to Haffey et al, issued Feb. 11, 1992; U.S. Pat. No. 4,509,949, to Huang et al, issued Apr. 5, 1985; U.S. Pat. No. 2,798,053, to Brown, issued Jul. 2, 1957; and in CTFA International Cosmetic Ingredient Dictionary, Fourth Edition, 1991, pp. 12 and 80.

Examples of commercially available carboxylic acid polymers useful herein include the carbomers, which are homopolymers of acrylic acid crosslinked with allyl ethers of sucrose or pentaerytritol. The carbomers are available as the Carbopol™ 900 series from B.F. Goodrich (e.g., Carbopol™ 954). In addition, other suitable carboxylic acid polymeric agents include copolymers of C₁₀₋₃₀ alkyl acrylates with one or more monomers of acrylic acid, methacrylic acid, or one of their short chain (i.e., C₁₋₄ alcohol) esters, wherein the crosslinking agent is an allyl ether of sucrose or pentaerytritol. These copolymers are known as acrylates/C₁₀₋₃₀ alkyl acrylate crosspolymers and are commercially available as Carbopol™ 1342, Carbopol™ 1382, Pemulen TR-1, and Pemulen TR-2, from B.F. Goodrich. In other words, examples of carboxylic acid polymer thickeners useful herein are those selected from carbomers, acrylates/C₁₀₋₃₀ alkyl acrylate crosspolymers, and mixtures thereof.

b) Crosslinked Polyacrylate Polymers

The compositions of the present disclosure can optionally contain crosslinked polyacrylate polymers useful as thickeners or gelling agents including both cationic and nonionic polymers, with the cationics being generally preferred. Examples of useful crosslinked nonionic polyacrylate polymers and crosslinked cationic polyacrylate polymers are those described in U.S. Pat. No. 5,100,660, to Hawe et al, issued Mar. 31, 1992; U.S. Pat. No. 4,849,484, to Heard, issued Jul. 18, 1989; U.S. Pat. No. 4,835,206, to Farrar et al, issued May 30, 1989; U.S. Pat. No. 4,628,078 to Glover et al issued Dec. 9, 1986; U.S. Pat. No. 4,599,379 to Flesher et al issued Jul. 8, 1986; and EP 228,868, to Farrar et al, published Jul. 15, 1987.

c) Polyacrylamide Polymers

The compositions of the present disclosure can optionally contain polyacrylamide polymers, especially nonionic polyacrylamide polymers including substituted branched or unbranched polymers. More preferred among these polyacrylamide polymers is the nonionic polymer given the CTFA designation polyacrylamide and isoparaffin and laureth-7, available under the Trade name Sepigel 305 from Seppic Corporation (Fairfield, N.J.).

Other polyacrylamide polymers useful herein include multi-block copolymers of acrylamides and substituted acrylamides with acrylic acids and substituted acrylic acids. Commercially available examples of these multi-block copolymers include Hypan SR150H, SS500V, SS500W, SSSA100H, from Lipo Chemicals, Inc., (Patterson, N.J.).

Compositions of the disclosure can therefore include desirable rheology modifiers such as carboxylic acid polymers, crosslinked polyacrylate polymers, polyacrylamide polymers, and mixtures thereof.

In certain embodiments, a pH modifier is utilized to activate the rheology modifier of the present disclosure. In such embodiments, a pH modifier can be added to an aqueous bath before or after a rheology modifier is added to adjust the pH of the aqueous bath so as to cause the bath to thicken.

As discussed above, a formulation including a rheology modifier can be added to water and with minimal agitation quickly create a thickened solution that has a mud-like consistency and feel. In this regard, suitable viscosities for a thickened bath can include a range that allows a consumer to have viscosities that are pleasing and appealing. In certain embodiments, viscosities can range from about 10,000 cps to about 300,000 cps.

It has been determined that, in certain embodiments, surfactants can aid in dispersing polymers more quickly in water which can be advantageous insofar as it can decrease the amount of rheology modifier needed to thicken water in a bath. In that regard, many surfactants are contemplated for use in the present disclosure. Examples of such surfactants are disclosed in McCutcheon's, Detergents and Emulsifiers, North American Edition (1997) published by Mc Publishing Co. of Glen Rock, N.J.

The surfactants can be present in an amount ranging from about 1% to about 99% by weight of the particular formulation of which the surfactant is a part, more specifically, from about 5% to about 70% by weight of the formulation, even more specifically, from about 10% to about 50% by weight of the formulation such that ionic character of the surfactant molecule is present below the level necessary to deswell the thickened composition.

The user can then soak his or her feet, hands, and/or body in this thickened solution to get the benefit derived from the formulation. It should be appreciated that the bath can be of any size and anything ranging from a bath tub, sink, bowl, or the like is contemplated by the present disclosure. When an acceptable amount of time has passed soaking, the user can deactivate the thickened solution and thin it to a water-like consistency by adding and agitating deswelling agent materials into the thickened solution. It should be understood that the user can remain in the bath while the thickened solution is deactivated or can exit from the bath during the deactivation step.

The deswelling agent can be present from about 0.01% to about 100% by weight of the particular formulation of which the deswelling agent is a part, more specifically, from about 0.1% to about 75% by weight of the formulation, even more specifically, from about 0.25% to about 50% by weight of the formulation. Examples of suitable deswelling agents that can used in a formulation to deactivate the rheology modifier thickened solution can include salts such sodium chloride, ammonium chloride, magnesium chloride, calcium chloride, and the like. In a preferred embodiment, the deswelling agent is sodium chloride and makes up 100% of the deactivating formulation.

Other examples of suitable deswelling agents include, but are not limited to, acids such as citric acid, lactic acid, glycolic acid and the like. In addition, basic deswelling agents can be utilized including sodium bicarbonate, sodium hydroxide, potassium hydroxide, and the like.

Other compounds that have an inherently high salt or ionic charge could also be utilized to de-swell the thickening system. Examples of such compounds include, but are not limited to surfactants that are anionic, nonionic, cationic, amphoteric, zwitterionic and the like. While not intending to be limited, such compounds can include sodium laureth sulfate, ammonium laureth sulfate, cocamidopropyl betaine, and the like.

In certain embodiments, if a pH modifier is used to activate the thickening system, the pH of the final solution in the bathtub can generally be from about 5 to about 7 as the preferred pH. If pH modification is used to deactivate the thickening system, pH can be adjusted higher or lower depending on chemistries used. For example, if salt is utilized, a general range from about 0% to about 5%, more specifically, from about 0.5% to about 4%, more specifically from about 1% to about 3%, can be used in relation to the final thickened bath.

Additionally, humectants such as sodium lactate and chelating agents such as disodium EDTA or tetrasodium EDTA could also be used. The above described deswelling agents are not intended to be limiting and other representative categories could effectively de-swell this thickening system as long as a change in ionic concentration is realized. In this regard, suitable viscosities for a thinned bath can, in some embodiments, include a range of viscosities similar to that of water, such as from about 1 cps to about 20,000 cps.

The fact that the formulation can be thinned easily to a water-thin liquid with ionic materials allows a user the benefit of an easy clean-up. The bath treatment can be poured down a drain or dumped into a toilet for easy disposal.

In addition to the above formulations for thickening and thinning bath treatment, numerous other beneficial chemistries could be added to any of the above-described formulations to provide the benefits derived from both a mud bath as well as a traditional bath formulation.

As described above, the bath treatment provides an attractive vehicle for the delivery of multiple benefits. The bath treatment can provide a user with formulations to benefit feet, hands, or any other part of the body. Examples of possible beneficial chemistries contemplated by the present disclosure include detoxifying agents, moisturizing agents, and soothing agents, but additional beneficial agents are also contemplated by the present disclosure.

Such beneficial chemistries can be present in amounts suitable depending upon which particular beneficial chemistries are utilized. For example, if antiaging peptides are used, they may be present such that the final concentration in the thickened bath would be from 0.0001% to about 5%. If humectants are used, they may be present such that the final concentration in the thickened bath would be from about 0.01% to about 25%.

In certain embodiments, detoxifying agents can be added to the bath treatment of the present disclosure. For example, in certain embodiments, detoxifying agents are added to one or more parts of the bath treatment formulation to make it appear and feel similar to a mud so a user can soak in a spa-like mud bath. In such embodiments, detoxifying agents can be added that have been described to pull toxins from the skin and provide a spa-like experience. Such detoxifying agents can include colorants and components of dirt that impart the look and color of a mud. These include but are not limited to clay, silt, ash, charcoal, activated charcoal, sand, FD&C dyes, pigments, diatomaceous earth, and the like.

Clays can be any cosmetically or pharmaceutically acceptable clay, either natural or synthetic. Examples of clays which can be used include, but are not limited to, Elquea Clay, Heilmoor Clay, Hakkaido Akan Clay, Manicouagun Clay, Morrocan Lava Clay, Tanakura Clay, Kaolin, Fullers Earth, Diatomaceous Earth. In certain embodiments, Kaolin is a preferred clay. The clays are mainly utilized to impart color to the composition.

In certain embodiments, moisturizing agents can be added to the bath treatment of the present disclosure. Moisturizing agents can be added to one or more parts of the bath treatment formulation to make a moisturizing lotion bath. This would give the user a spa-like experience of a relaxing soak in skin beneficial hydrating ingredients and allow them an easy, convenient alternative to applying lotion while relieving stress. Various components could be added to provide this benefit including, but not limited to moisturizing agents which impart some moisturizing benefit to the skin. These include, but are not limited to humectants such as glycerin, glycols, and sorbitol; synthetic oils such as mineral oil and petrolatum; natural oils such as sunflower oil, jojoba oil, and safflower oil; silicones such as dimethicone, cyclomethicone; esters such as isopropyl palmitate, caprylic/capric triglyceride; butters such as cocoa butter, coffee butter, and shea butter; barrier ingredients such as fatty acids, fatty alcohols, and waxes.

In certain embodiments, soothing agents can also be added to the bath treatment formulation of the present disclosure to provide a user a spa-like experience of a relaxing soak while helping to minimize symptoms such as itching, redness, inflammation, and the like and allow them an easy, convenient alternative to applying topical treatments while relieving stress. Various soothing agents could be added to provide this benefit including, but not limited to SymCalmin from Symrise, oat derived ingredients such as colloidal oatmeal, bisabolol, allantoin, herbal extracts such as chamomile extract and the like.

The bath can also serve additional purposes and can be an inexpensive and effective way for a consumer to pamper oneself. For example, in certain embodiments, one can soak for a length of time and have a relaxing spa experience at home by exerting little effort in creating the thickened bath. Additionally, after soaking and emerging from the tub, a consumer's core body temperature, which can be elevated in warm water, can suddenly drop causing heart rate and breathing to slow. This can result in making a consumer feel drowsy and relaxed. Additionally, adding essential oils and/or herbs with sedative properties can boost such a calming effect. The heat of a warm bath can also have therapeutic benefits to soothe tight/sore muscles by increasing circulation and boosting the blood flow to such muscles. This in turn loosens the muscles and aids in healing. The warm bath can also add a benefit for relief of sore joints from stress or arthritis. Ingredients that could be added to the bath to increase the soothing benefit could be external analgesics, neurosensory agents (menthol, menthyl lactate, camphor), essential oils of peppermint, and/or various aromatherapies as would be known in the art. Additionally, it is known in literature that salts such as Epsom and mineral salts are high in magnesium which is a known muscle relaxer that can loosen muscles further. In certain embodiments, such components can be added in the deswelling step and the consumer could continue to soak in the deswelled composition for even further benefits.

The above examples are not meant to be all-inclusive and soothing agents from each example could also be used in another example to provide secondary benefit. An example of this would be the inclusion of moisturizers in the mud bath to make a product that was not just detoxifying, but moisturizing as well.

Additionally, normal additives from the CTFA Dictionary could be added to these embodiments to add to the overall functionality of the products. For instance, certain components can be added to the bath treatment that are suitable for contact with human keratinous tissue. Hence, such additional components do out have undue toxicity, incompatibility, instability, allergic response, and the like within the scope of sound medical judgment.

The CTFA Cosmetic Ingredient Handbook, Second Edition (1992) describes a wide variety of nonlimiting cosmetic and pharmaceutical ingredients which are suitable for use in the bath treatments of the present disclosure. Examples of these ingredient classes include: abrasives, absorbents, aesthetic components such as fragrances, pigments, colorings/colorants, essential oils, skin sensates, astringents (clove oil, menthol, camphor, eucalyptus oil, eugenol, menthyl lactate, witch hazel distillate), anti-acne agents, anti-caking agents, antifoaming agents, antimicrobial agents, antioxidants, binders, biological additives, buffering agents, bulking agents, chelating agents, chemical additives, colorants, cosmetic astringents, cosmetic biocides, denaturants, drug astringents, external analgesics, film formers, opacifying agents, pH adjusters, propellants; reducing agents, sequestrants, skin bleaching and lightening agents (e.g., hydroquinone, kojic acid, ascorbic acid, magnesium ascorbyl phosphate, ascorbyl glucosamine), skin-conditioning agents (e.g., humectants, including miscellaneous and occlusive), skin soothing and/or healing agents (e.g., panthenol and panthenol derivatives), aloe vera, pantothenic acid, pantothenic acid derivatives, allantoin, bisabolol, and dipotassium glycyrrhizinate, skin treating agents, sunscreens, thickeners, and vitamins and derivatives thereof.

The bath treatment of the present disclosure can be provided in any suitable dispenser as would be known in the art such as bottles, tubes, jars, sprays or any other viable form. Additionally, any of the above-described formulations or components can be provided in any suitable liquid form, emulsions or otherwise. In certain embodiments, the formulations are used in connection with anhydrous liquids, which as used herein refers to liquids with less than about 5% by weight water, preferably less than 1% by weight water.

In the interests of brevity and conciseness, any ranges of values set forth in this specification are to be construed as written description support for claims reciting any sub-ranges having endpoints which are whole number values within the specified range in question. By way of a hypothetical illustrative example, a disclosure in this specification of a range of 1-5 shall be considered to support claims to any of the following sub-ranges: 1-4; 1-3; 1-2; 2-5; 2-4; 2-3; 3-5; 3-4; and 4-5.

The disclosure will be clarified by the following data, which is intended to be purely exemplary of the disclosure. In the experimental data provided below, some of the results have been obtained through certain embodiments of the present disclosure.

### EXAMPLE 1

The following example measured the impact of formulation viscosity on drainage attributes.

### Materials:

Funnel with an opening approximately ¼″ in diameter
Glass bottle capable of holding ˜100 g of solution and with a neck that the funnel can fit into easily
(1) 1500 mL glass Beaker
(5) 200 mL glass beakers
Plastic spatula (for stirring)

### Mud Soak Formulation:

Phase 1:
15 g Rapithix A60 (Sodium Polyacrylate, Hydrogenated Polydecene, Trideceth-6)

Phase 2:
13 g Golden Moor Mud (Heilmoor Mud)
1.5 g Mackam 35UL CAPB (Cocamidopropyl Betaine)

### Procedure:

### Making Thickened Mud:

1) Add 12.00 grams of Phase 2 to 1500 mL beaker and fill to 1000 mL with tap water.
2) While mixing with the plastic spatula, add Phase 1
3) Mix until thickened

### Deactivating the Mud Polymer System:

1) Weigh out 200 grams of thickened mud
2) Add 2 grams of sodium chloride
3) Mix until salt is well dispersed and formulation becomes thin

### Test Procedure:

1) Take viscosity measurement of formulation

Water 2.50 cps (LV2 Spindle, 30 RPM)

Thickened Mud 200,000 cps (LVT4 0.6 RPM)

Mud+1% Salt 9 cps (LV2 30 RPM)
2) Pre-weigh a beaker and pour 100.00 g of formulation into it

Water 100.07 g

Thickened Mud 100.01 g

Mud+1% Salt 100.02 g
3) Pre-weigh the bottle before adding formulation

Bottle (Water) 152.68 g

Bottle (Thickened Mud) 152.67 g

Bottle (Mud+1% Salt) 152.68 g
4) Pour the formulation from the beaker into the funnel/bottle.
5) Remove the funnel and weigh the bottle

Water+Bottle 252.58 g,

Thickened Mud+Bottle 152.67 g,

Mud+1% Salt+Bottle 252.03
6) Subtract the weight of the bottle before adding the formulation from the weight of the bottle after passing the formulation through the funnel into the bottle to determine the amount of formulation able to drain through the funnel

Water 252.58 g−152.68 g=99.90 g

Thickened Mud 152.67 g−152.67 g=0.00 g

Mud+1% Salt 252.03 g−152.68 g=99.35 g
7) Subtract the amount of formulation collected in the bottle from the original amount of formulation added to the funnel/bottle to determine formulation loss to the original beaker and funnel

Water 100.07 g−99.90 g=0.17 g

Thickened Mud 100.01 g−0.00 g=100.01 g

Mud+1% Salt 100.02 g−99.35 g=0.67 g

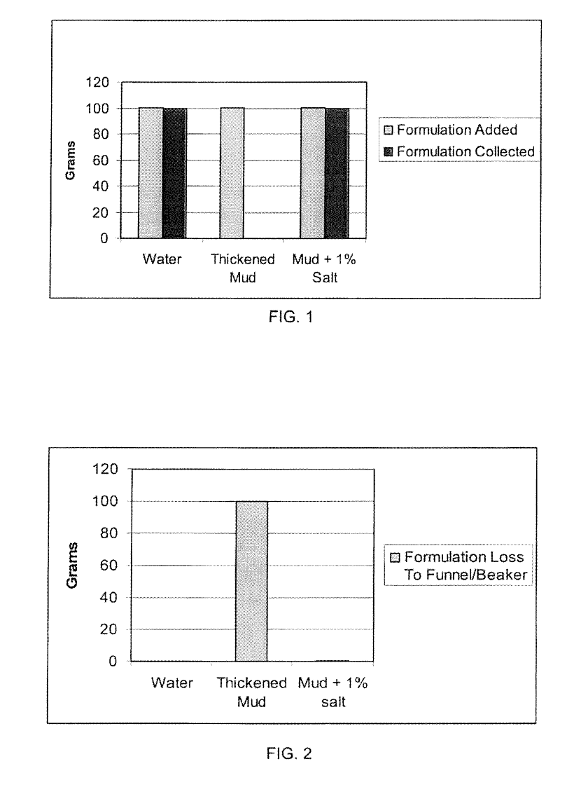

Test results can be found in FIGS. 1 and 2 and indicate that formulation viscosity can affect drainage.

### EXAMPLE 2

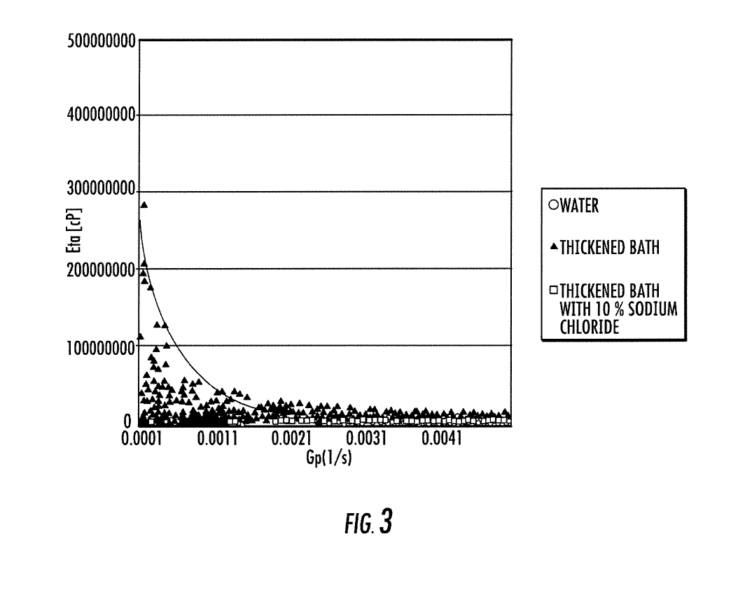

FIG. 3 graphs rheology data of water, a bath consisting of 5% RapiThix (remaining water), and a bath consisting of 5% RapiThix (remaining water) in which 10% sodium chloride was added. Test results indicate that sodium chloride added to 5% RapiThix decreases viscosity. The figure depicts that upon addition of the deswelling agent (i.e. the salt), the rheology of the thickened bath is similar to that of water in that the rate of shear does not dramatically affect the measured viscosity. This is dramatically different than the thickened bath in which the viscosity decreases substantially with shear.

Examples 3-8 show various embodiments of the present disclosure. The following examples have percent weight indicated for the percentage of each ingredient within the thickening/swelling formulation.

### EXAMPLE 3

| Trade Name | INCI | % wt |
|---|---|---|
| Carbopol Aqua SF-1 | Acrylates Copolymer | 44.69 |
| Polymer | | |
| Neolone 950 | Methylisothiazolinone | 0.56 |
| Liposilt Green | Silt | 10.06 |
| Sunflower Oil | Helianthus Annus (Sunflower) Seed Oil | 27.49 |
| Hetester PHA | Propylene Glycol Isoceteth-3 Acetate | 4.47 |
| Arlatone T | PEG-40 Sorbitan Peroleate | 2.23 |
| Cognis IPP | Isopropyl Palmitate | 8.94 |
| Tocopheryl Acetate | Tocopheryl Acetate | 0.22 |
| Mandarin & Ginger | Fragrance | 1.34 |
| Flower 178470B | | |

Add concentrate to water.
Add basic pH adjuster until pH of bathtub
is between 5-7 to thicken
Add salt until polymeric system breaks
and viscosity approaches that of water

### EXAMPLE 4

| Trade Name | INCI | Wt % |
|---|---|---|
| Carbopol Ultrez 21 | Acrylates/C10-30 Alkyl Acrylate | 4.50 |
| | Crosspolymer | |
| Neolone 950 | Methylisothiazolinone | 0.05 |
| Liposilt Green | Silt | 6.53 |
| Florasun 90 | Helianthus Annus (Sunflower) Seed Oil | 47.96 |
| Hetester PHA | Propylene Glycol Isoceteth-3 Acetate | 7.18 |
| Cognis IPP | Isopropyl Palmitate | 22.86 |
| Phenonip XB | Phenoxyethanol, Methylparaben, | 1.02 |
| | Ethylparaben, Propylparaben | |
| Arlatone T | PEG-40 Sorbitan Peroleate | 7.72 |
| Mandarin & Ginger | Fragrance | 2.18 |
| Flower 178470B | | |

Add concentrate to water.
Add basic pH adjuster until pH of bathtub is between 5-7 to thicken
Add salt until polymeric system breaks and viscosity approaches that of water

### EXAMPLE 5

| Trade Name | INCI | Wt % |
|---|---|---|
| Aristoflex AVC | Ammonium Acryloyldimethyltaurate/VP | 17.97 |
| | Copolymer | |
| Celite Snow Floss | Diatomaceous Earth | 7.41 |
| Phenonip XB | Phenoxyethanol, Methylparaben, | 0.79 |
| | Ethylparaben, Propylparaben | |
| Sunflower Oil | Helianthus Annus (Sunflower) Seed Oil | 67.22 |
| Hetester PHA | Propylene Glycol Isoceteth-3 Acetate | 3.36 |
| Arlatone T | PEG-40 Sorbitan Peroleate | 2.24 |
| Mandarin & Ginger | Fragrance | 1.01 |
| Flower 178470B | | |

Add concentrate to water
Agitate to thicken.
Add salt until polymeric system breaks
and viscosity approaches that of water

### EXAMPLE 6

| Trade Name | INCI | Wt % |
|---|---|---|
| Rapithix A-60 | Sodium Polyacrylate, Hydrogenated | 96.3 |
| | Polydecene, Trideceth-6 | |
| Ochre Pink Clay | Clay | 3.2 |
| Charcoal | Charcoal | 0.5 |

Add concentrate to water
Agitate to thicken.
Add salt until polymeric system breaks
and viscosity approaches that of water

### EXAMPLE 7

Product Name: One Phase Mud Foot Soak

| Trade Name | INCI Name | % wt |
|---|---|---|
| Sepigel 501 | C13-14 Isoparaffin, Mineral Oil, Sodium | 58.83 |
| | Polyacrylate, Polyacrylamide, | |
| | Polysorbate 85 | |
| Liposilt Black | Silt | 10.70 |
| Kathon CG | Methylchloroisothizalone and | 2.1400 |
| | methylisothizolone | |
| Disodium EDTA | Disodium EDTA | 1.0700 |
| Sunflower Oil | Sunflower Oil | 16.71 |
| Heterester PHA | Propylene Glycol Isoceteth-3 Acetate | 2.67 |
| Stepan IPM | Isopropyl Myristate | 5.35 |
| Tocopeheryl Acetate | Tocopheryl Acetate | 0.13 |
| Mandarin, Ginger | Fragrance | 0.53 |
| and Flowers | | |
| Fragrance | | |
| Arlatone T | PEG-40 Sorbitan Peroleate | 1.34 |
| BHT | BHT | 0.53 |

Add concentrate to water
Agitate to thicken.
Add salt until polymeric system breaks
and viscosity approaches that of water

### EXAMPLE 8

Product Name: 1 Phase Mud Foot Soak

| Trade Name | INCI Name | % wt |
|---|---|---|
| Butylene Glycol | Butylene Glycol | 14.48 |
| Ochre Red Clay | Kaolin | 6.42 |
| Sepigel 501 | C13-14 Isoparaffin, Mineral Oil, Sodium | 77.18 |
| | Polyacrylate, Polyacrylamide, Polysorbate 85 | |
| Frescolate | Methyl Lactate | 0.96 |
| Liposilt Green | Silt | 0.96 |

Add concentrate to water
Agitate to thicken.
Add salt until polymeric system breaks
and viscosity approaches that of water

The formulations described in the above examples are intended to be added to an amount of water such that the final percentage of polymer within the thickened bath solution is within the effective range of the polymer to provide viscosity as taught. For example, Example 6 can be added to a bathtub of water (˜25-30 gallons) in an amount of 1-2 liters to provide the desired thickening effect.

These and other modifications and variations to the present disclosure may be practiced by those of ordinary skill in the art, without departing from the spirit and scope of the present disclosure, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the disclosure so further described in such appended claims.

1. A bath treatment kit comprising:
a first formulation, the first formulation including a rheology modifier, the rheology modifier being present in a liquid, the rheology modifier capable of thickening an aqueous bath;
a second formulation, the second formulation including a deswelling agent, the deswelling agent capable of thinning an aqueous bath previously thickened by the rheology modifier.

2. The bath treatment kit of claim 1, wherein the first formulation further comprises a beneficial skin chemistry.

3. The bath treatment kit of claim 2, wherein the beneficial chemistry comprises a detoxifying agent, a moisturizing agent, a soothing agent, or mixtures thereof.

4. The bath treatment kit of claim 1, wherein the rheology modifier comprises a polyacrylate polymer, a polyacrylamide polymer, clay or mixtures thereof.

5. The bath treatment kit of claim 1, wherein the deswelling agent is capable of changing the ion concentration of the aqueous bath previously thickened by the rheology modifier.

6. The bath treatment kit of claim 1, wherein the rheology modifer is present in the first formulation in an amount ranging from about 1 to about 99 weight percent.

7. The bath treatment kit of claim 1, wherein the deswelling agent is present in the second formulation in an amount ranging from about 0.01 to about 100 weight percent.

8. The bath treatment kit of claim 1, further comprising an additional formulation, the additional formulation including a surfactant.

9. The bath treatment kit of claim 1, further comprising an additional formulation, the additional formulation including a pH modifier.

10. The bath treatment kit of claim 1, wherein the liquid of the first formulation comprises an anhydrous liquid.

11. The bath treatment kit of claim 1, further comprising a means for dispensing the first formulation and second formulation in an aqueous bath.

12. A method of using a bath treatment kit comprising the steps of:
adding a first formulation to a water container, the first formulation including a rheology modifier, the rheology modifier being present in a liquid, the rheology modifier thickening a portion of the water in the water container; and
adding a second formulation to a water container, the second formulation including a deswelling agent, the deswelling agent capable of thinning the water in the water container previously thickened by the rheology modifier.

13. The method of claim 12, further comprising the step of immersing a part of the body in the water container and removing such part of the body from the water container.

14. The method of claim 12, further comprising the step of adding a surfactant to the water container.

15. The method of claim 12, wherein the first formulation further comprises a beneficial skin chemistry.

16. The method of claim 15, wherein the beneficial chemistry comprises a detoxifying agent, a moisturizing agent, a soothing agent, or mixtures thereof.

17. The method of claim 12, wherein the rheology modifier comprises a polyacrylate polymer, a polyacrylamide polymer, clay or mixtures thereof.

18. The method of claim 12, wherein the deswelling agent is capable of changing the ion concentration of the aqueous bath previously thickened by the rheology modifier.

19. The method of claim 12, wherein the rheology modifer is present in the first formulation in an amount ranging from about 1 to about 99 weight percent.

20. The method of claim 12, wherein the deswelling agent is present in the third formulation in an amount ranging from about 0.01 to about 100 weight percent.

21. The method of claim 12, wherein the liquid of the first formulation comprises an anhydrous liquid.

22. The method of claim 14, wherein the surfactant is added to the water container before the first formulation.

23. The method of claim 12, further comprising the step of adding a pH modifier to the water container.

24. The method of claim 23, wherein the pH modifier is added to the water container after the first formulation is added to the water container but before the second formulation is added to the water container.

25. The method of claim 12, further comprising the steps of adding a pH modifier to the water container and adding a surfactant to the water container, wherein the surfactant is added to the water container before the first formulation is added to the water container, and the pH modifier is added to the water container after the first formulation is added to the water container but before the second formulation is added to the water container.
